(19) United States
(12) Patent Application Publication
(10) Pub. No.: US 2026/0337596 A1
(43) Pub. Date: Oct. 29, 2026

(54) BENTONITE-AMINO ACID COMPLEX, BENTONITE-PROTEIN COMPLEX AND METHOD OF PREPARING THEREOF

(71) Applicant: KOREA INSTITUTE OF GEOSCIENCE AND MINERAL RESOURCES, Daejeon (KR)

(72) Inventors: Jaehwan KIM, Pohang-si (KR); Sung Man SEO, Pohang-si (KR); Daeyoung KIM, Geumsan-gun (KR); ll-Mo KANG, Seoul (KR); Sang Gun NO, Pohang-si (KR); Jongkyu PARK, Pohang-si (KR)

(73) Assignee: KOREA INSTITUTE OF GEOSCIENCE AND MINERAL RESOURCES, Daejeon (KR)

(21) Appl. No.: 19/191,309

(22) Filed: Apr. 28, 2025

(51) Int. Cl. C12N 11/14; C12N 9/64; C12N 9/76

(52) U.S. Cl. CPC C12N 11/14; C12N 9/6427; C12N 9/6481; C12Y 304/21004; C12Y 304/23

(57) ABSTRACT

Provided is a bentonite-amino acid complex including a bentonite; and an amino acid. The complex is formed by immobilizing the amino acid on the bentonite, and the amino acid is a hydrophilic amino acid.

### BACKGROUND

### 1. Field

The present disclosure relates to a bentonite-amino acid complex, a bentonite-protein complex, and a method of preparing the same.

### 2. Description of Related Art

Bentonite is a type of layered silicate mineral that is created by the transformation of volcanic ash, and is mainly composed of smectite minerals, and also of clay from the mineral montmorillonite. Its microstructure is a stacked flat plate structure. Each flat plate is composed of three sandwiched layers: a central octahedral alumina (Al₂O₃) layer and two tetrahedral silica (SiO₂). The properties of bentonite include large interlayer spaces, cation exchange capacity, antimicrobial activity, drug-transport and release, swelling and hygroscopicity, gel formation, and the ability to act as cell-centered adsorption sites that enhance cell attachment and cell proliferation. Currently, the utilization of these clay minerals as pharmaceutical raw materials is low in Korea. Although there are significant reserves of bentonite in Korea, bentonite contains impure minerals such as quartz, feldspar, mica, iron oxide, and silica hydrate, and has a low content of montmorillonite, and thus is mainly used for foundry sand, binder, and civil engineering. However, in recent years, attempts have been made to develop basic pharmaceutical materials through transforming mineral resources into pharmaceutical raw materials, and in particular, for this purpose, bentonite is attracting attention as it is expected that bentonite will be utilized as a high value-added mineral resource in the future.

### SUMMARY

The technical goal to be achieved by the present disclosure is to provide a bentonite-amino acid complex, a bentonite-protein complex, and a method of preparing the same, which may be synthesized under various salt concentrations and pH conditions while a stable bond and enzymatic activity in the complexes is maintained.

However, the technical goals to be achieved by the present disclosure are not limited to those described above, and other goals not mentioned above may be clearly understood by one of ordinary skill in the art from the following description.

An aspect of the present disclosure provides a bentonite-amino acid complex including a bentonite; and an amino acid, wherein the complex is formed by immobilizing the amino acid on the bentonite, and the amino acid is a hydrophilic amino acid.

In one embodiment, the amino acid may include any one or more amino acids selected from the group consisting of arginine, histidine, lysine, aspartic acid, glutamic acid, serine, threonine, asparagine, glutamine, cysteine, glycine, and proline.

In one embodiment, the amino acid may have a positive charge.

In one embodiment, the amino acid may be a histidine.

In one embodiment, the bentonite-amino acid complex may maintain a bond between the bentonite and the amino acid at pH 1 to pH 10 for more than 72 hours.

In one embodiment, the bentonite-amino acid complex may maintain a bond between the bentonite and the amino acid for 8 hours at a salt concentration of 4.0 M or less.

One aspect of the present disclosure provides a bentonite-protein complex including a bentonite; and a protein, wherein the complex is formed by immobilizing a protein on the bentonite, and the protein is a protein including histidine.

In one embodiment, the bentonite-protein complex may include no more than 2000 μg of the protein per 10 mg of the bentonite.

In one embodiment, the protein is trypsin, and the bentonite-protein complex may include no more than 2000 μg of the trypsin per 10 mg of the bentonite.

In one embodiment, the protein may include any one or more proteins selected from the group consisting of trypsin, pepsin, human serum albumin and bovine serum albumin (BSA).

In one embodiment, the protein is bovine serum albumin (BSA), and the bentonite-protein complex may include less than 1000 μg of bovine serum albumin (BSA) per 10 mg of the bentonite.

In one embodiment, the bentonite-protein complex may maintain a bond between the bentonite and the protein at pH 1 to pH 10 for more than 72 hours.

In one embodiment, the bentonite-protein complex may maintain a bond between the bentonite and the protein for 8 hours at a salt concentration of 4.0 M or less.

In one embodiment, the protein is trypsin or pepsin, and the bentonite-protein complex may be used as a digestive enzyme.

One aspect of the present disclosure provides a method of preparing a bentonite-amino acid complex, including: washing a bentonite; preparing a buffer containing an amino acid; mixing the bentonite and the buffer to bind the bentonite and the amino acid; and centrifuging the mixed solution to separate unbound amino acids.

In one embodiment, the amino acid may be a hydrophilic amino acid.

In one embodiment, the amino acid may have a positive charge.

In one embodiment, the amino acid may be a histidine.

One aspect of the present disclosure provides a method of preparing a bentonite-protein complex, including: washing a bentonite; preparing a buffer containing a protein; mixing the bentonite and the buffer to bind the bentonite and the protein; and centrifuging the mixed solution to separate unbound proteins.

In one embodiment, the protein may be a protein including histidine.

In one embodiment, the protein may include any one or more proteins selected from the group consisting of trypsin, pepsin, human serum albumin and bovine serum albumin (BSA).

Additional aspects of embodiments will be set forth in part in the description which follows and, in part, will be apparent from the description, or may be learned by practicing the disclosure.

The bentonite-amino acid complex and bentonite-protein complex of the present disclosure may be synthesized under various salt concentrations and pH conditions while a stable bond is maintained, and the enzymatic activity of the protein may be maintained even while the protein is bound within the complexes. Hence, the bentonite-amino acid complex and bentonite-protein complex of the present disclosure have the effect of transporting proteins, including digestive enzymes, to the sites under various conditions in the body, and have the effect of enabling the enzymatic action of the protein within the complex in the various environments in the body.

It should be understood that the effects of the present disclosure are not limited to the above-described effects, but are to be construed as including all effects that may be inferred from the configurations and features described in the following description or claims of the present disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A to 1D show the results of SDS-PAGE and fluorescence imaging for the bentonite-amino acid complex synthesized at pH 7.4 and 9.0 according to one embodiment of the present disclosure;

FIGS. 2A and 2B show the results of SDS-PAGE and Western blot analysis for the bentonite-amino acid complex stored at pH 7.4 and 9.0 according to one embodiment of the present disclosure;

FIG. 3 shows the result of SDS-PAGE for the bentonite-protein complex stored at pH 1.2 according to one embodiment of the present disclosure;

FIG. 4 shows the results of SDS-PAGE and Western blot analysis for the bentonite-amino acid complex stored at various salt concentrations according to one embodiment of the present disclosure;

FIGS. 5A to 5D show the results of SDS-PAGE and Western blot analysis to determine the enzymatic activity of trypsin bound to the bentonite according to one embodiment of the present disclosure;

FIG. 6 shows the result of the X-ray diffraction analysis of a bentonite-trypsin complex;

FIGS. 7A and 7B show the result of SDS-PAGE to determine the protein binding capacity of bentonite;

FIG. 8 shows the results of SDS-PAGE and Western blot analysis of a bentonite-HSA complex in accordance with one embodiment of the present disclosure; and

FIGS. 9A and 9B show the results of SDS-PAGE and Western blot analysis of a bentonite-pepsin complex in accordance with one embodiment of the present disclosure.

### DETAILED DESCRIPTION

Hereinafter, embodiments will be described in detail with reference to the accompanying drawings. However, various alterations and modifications may be made to the embodiments. Thus, the scope of the present application is not to be construed as limited or circumscribed by such embodiments. With respect to the embodiments, it will be understood that the scope of the present disclosure shall include all changes, equivalents, and replacements.

The terminology used herein is for the purpose of describing particular embodiments only and is not intended to be limiting. The singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as is commonly understood by one of ordinary skill in the art to which the examples belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

In addition, in the descriptions of the embodiments making reference to the accompanying drawings, like reference numerals refer to like constituent elements and any repeated description related thereto has been omitted. In the descriptions of the embodiments, any detailed description of well-known related art has been omitted where it is deemed that such description will cause ambiguous interpretation of the present disclosure.

In addition, terms such as first, second, A, B, (a), (b), and the like may be used to describe components of the embodiments. These terms are used only for the purpose of discriminating one constituent element from another constituent element, and do not limit the nature, the sequences, or the orders of the constituent elements.

A constituent element, which has the same common function as a constituent element included in any one embodiment, will be referred-to by the name that is used for the element in the descriptions of other embodiments. Unless stated otherwise, the description of one embodiment may be applicable to other embodiments and thus, duplicated descriptions have been omitted for conciseness.

In the present specification, when a certain part “includes” a certain component, this indicates that the part may further include another component and not that the part excludes another component.

An aspect of the present disclosure provides a bentonite-amino acid complex including a bentonite; and an amino acid, wherein the complex is formed by immobilizing the amino acid on the bentonite, and the amino acid is a hydrophilic amino acid.

In one embodiment, the amino acid may include any one or more amino acids selected from the group consisting of arginine, histidine, lysine, aspartic acid, glutamic acid, serine, threonine, asparagine, glutamine, cysteine, glycine, and proline, and may preferably be an amino acid having a positive charge, more preferably a histidine.

The bentonite-amino acid complex according to one embodiment of the present disclosure may be formed by binding a bentonite to an amino acid at a pH of 1 to 10, preferably at a pH of 1.2 to 9, more preferably at a pH of 7.4 to 9, wherein the binding may involve immobilizing an amino acid on the bentonite. The complex formed may be one that maintains the bond between bentonite and amino acid at a pH of 1 to 10, preferably at a pH of 1.2 to 9, for 72 hours or more, or 48 hours or more, or 24 hours or more, and may also be one that maintains the bond between bentonite and amino acid, even at a salt concentration of NaCl of 4.0 M or less, or 2.5 M or less for 8 hours.

One aspect of the present disclosure provides a bentonite-protein complex including a bentonite; and a protein, wherein the complex is formed by immobilizing a protein on the bentonite, and the protein is a protein including histidine.

In one embodiment, the protein may include any one or more proteins selected from the group consisting of trypsin, pepsin, human serum albumin and bovine serum albumin (BSA).

In one embodiment, the bentonite-protein complex may include no more than 2000 μg of the protein, preferably no more than 1000 μg of the protein, per 10 mg of the bentonite. In particular, when the protein is trypsin, the trypsin may be included in an amount of no more than 2000 μg, preferably no more than 1000 μg, per 10 mg of the bentonite, and when the protein is bovine serum albumin (BSA), the bovine serum albumin (BSA) may be included in an amount of less than 1000 μg, preferably less than 500 μg, per 10 mg of the bentonite. Depending on the type of protein, there may be differences in the amount of protein that may bind to the bentonite, which may be due to the size of the protein that binds to the bentonite.

The bentonite-protein complex according to one embodiment of the present disclosure may be formed by binding bentonite to a protein at a pH of 1 to 10, preferably at a pH of 1.2 to 9, more preferably at a pH of 7.4 to 9, wherein the binding may involve immobilizing a protein on bentonite. The complex formed may be one that maintains the bond between bentonite and protein at a pH of 1 to 10, preferably at a pH of 1.2 to 9, for 72 hours or more, or 48 hours or more, or 24 hours or more, and may also be one that maintains the bond between bentonite and protein, even at a salt concentration of NaCl of 4.0 M or less, or 2.5 M or less for 8 hours. The bentonite-protein complex according to the present disclosure may maintain a stable bond even in a wide range of pH and salt concentrations as described above, and in particular, the bentonite-protein complex may maintain its bond even in the acidic conditions similar to those in the stomach, pH 1.2; the biological pH of the human body, pH 7.4; and the alkaline conditions higher than the pH of the human body, pH 9, so that the protein bound to the bentonite may be stably transported to internal organs of the body.

In one embodiment of the present disclosure, a bentonite-protein complex may be provided, wherein the protein is trypsin or pepsin; and wherein the trypsin or pepsin of the complex may still retain its original enzymatic activity, so that the trypsin or pepsin bound to the bentonite may act as a digestive enzyme that decomposes proteins even in the form of a complex.

One aspect of the present disclosure provides a method of preparing a bentonite-amino acid complex, including: washing a bentonite; preparing a buffer containing an amino acid; mixing the bentonite and the buffer to bind the bentonite and the amino acid; and centrifuging the mixed solution to separate unbound amino acids. The amino acid may be a hydrophilic amino acid, preferably may be an amino acid having a positive charge, and more preferably may be a histidine.

One aspect of the present disclosure provides a method of preparing a bentonite-protein complex, including: washing a bentonite; preparing a buffer containing a protein; mixing the bentonite and the buffer to bind the bentonite and the protein; and centrifuging the mixed solution to separate unbound proteins. In one embodiment, the protein may be a protein including histidine, or the protein may include any one or more proteins selected from the group consisting of trypsin, pepsin, human serum albumin and bovine serum albumin (BSA).

In one embodiment, the buffer may have a pH of 1 to 10, preferably a pH of 1.2 to 9, more preferably a pH of 7.4 to 9, and thus the bentonite-protein complex according to the present disclosure may be stably synthesized in the described pH ranges.

Hereinafter, the present disclosure will be described in more detail with reference to the following examples. The following examples are provided only for the purpose of illustrating the present disclosure and are not intended to limit the scope of the present disclosure.

### EXAMPLES

### 1. Preparation Example

### Example 1

10 mg of bentonite was washed twice with 1 ml of Tris-Cl buffer (pH 7.4) to equilibrate the particles. A solution was prepared by adding 20μg of purified green fluorescent protein marker-tagged histidine (His-GFP) to 100 μl of Tris-Cl buffer at pH 7.4. The bentonite was mixed with the above mentioned His-GFP-added buffer, and then the mixture was stirred using a twister/vortex mixer at 4 °C for 2 hours to bind the His-GFP to the bentonite. The mixture was then centrifuged at 5000 G for 5 minutes to separate the mixture into a supernatant containing unbound His-GFP and a pellet containing the bentonite-amino acid complex. The separated pellet was washed four times with ice-cold Tris-Cl buffer at pH 7.4 to remove loosely bound His-GFP from the bentonite-amino acid complex. For subsequent analysis, the bentonite-amino acid complex was added to SDS sample buffer and this mixed buffer solution was boiled to separate His-GFP from the bentonite. The supernatant containing unbound His-GFP (UB), amino acid bound to bentonite (B), and the pellet-washing solution (W) were extracted at each step and stored.

### Example 2

The bentonite-amino acid complex was prepared in the way it was prepared in Example 1 presented above, except that Tris-Cl buffer at pH 9.0 was used.

### Example 3

The bentonite-trypsin complex was prepared in the way it was prepared in Example 1 presented above, except that trypsin was used as protein instead of His-GFP.

### Example 4

10 mg of bentonite was mixed with PBS buffer (pH 7.4) containing 50 μg of trypsin. Then, the mixture was stirred using a twister/vortex mixer at 4 °C for 1 hours to bind the protein to the bentonite. The mixture was then centrifuged at 5000 G for 5 minutes to separate the mixture into a supernatant containing unbound proteins and a pellet containing the bentonite-protein complex. The separated pellet was washed two times with PBS buffer to remove loosely bound proteins from the bentonite-protein complex. For subsequent analysis, the bentonite-protein complex was added to SDS sample buffer and this mixed buffer solution was boiled to separate the protein from the bentonite. The supernatant(S) and proteins bound to the bentonite (B) were extracted at each step and stored.

### Example 5

The bentonite-protein complex was prepared in the way it was prepared in Example 4 presented above, except that 100 μg of trypsin was added as protein.

### Example 6

The bentonite-protein complex was prepared in the way it was prepared in Example 4 presented above, except that 500 μg of trypsin was added as protein.

### Example 7

The bentonite-protein complex was prepared in the way it was prepared in Example 4 presented above, except that 1000 μg of trypsin was added as protein.

### Example 8

The bentonite-protein complex was prepared in the way it was prepared in Example 4 presented above, except that 50 μg of bovine serum albumin (BSA) was added as protein.

### Example 9

The bentonite-protein complex was prepared in the way it was prepared in Example 4 presented above, except that 100 μg of bovine serum albumin (BSA) was added as protein.

### Example 10

The bentonite-protein complex was prepared in the way it was prepared in Example 4 presented above, except that 500 μg of bovine serum albumin (BSA) was added as protein.

### Example 11

The bentonite-protein complex was prepared in the way it was prepared in Example 4 presented above, except that 1000 μg of bovine serum albumin (BSA) was added as protein.

### Example 12

The bentonite-protein complex was prepared in the way it was prepared in Example 1 presented above, except that 50 mg of bentonite was added and 1 mg of human serum albumin (BSA) was added as protein instead of His-GFP.

### Example 13

The bentonite-protein complex was prepared in the way it was prepared in Example 1 presented above, except that 50 mg of bentonite was added and 1 mg of pepsin was added as protein instead of His-GFP.

### 2. Experimental Example 1: SDS-PAGE and Western Blot Analysis of Bentonite-Amino Acid Complexes Prepared at Different pHs

In order to confirm whether the bentonite-amino acid complex was properly formed by the preparation procedure of Examples 1 and 2 presented above, SDS-PAGE and Western blot analyses were performed on the samples of supernatant (UB), pellet (B), and washing solution (W) that were extracted and stored during the preparation procedure of Examples 1 and 2 presented above. A description of the analysis methods follows.

### SDS-PAGE

The samples were separated by molecular weight using 12% or 15% SDS/PAGE. The gel was then stained with 0.25% CBB R-250 (AMRESCO, cat. no: 6104-59-2) in a solution containing 45% methanol and 10% glacial acetic acid.

### Western Blot Analysis

The samples were separated by molecular weight using 12% or 15% SDS/PAGE. The proteins in the gel were then blotted onto a filter membrane, which was blocked for 1 hour with 5% non-fat skim milk dissolved in TBST buffer (20 mM Tris-Cl, pH 7.5, 150 mM NaCl, 0.1% Tween-20). As primary antibodies, mouse anti-GPF (Living colors, Takara) or mouse anti-His (Novus Bio, USA) antibodies were diluted 1:1000-1:5000 in a TBST solution containing 5% non-fat skim milk. The filter membrane was incubated overnight in the diluted solution, and the primary antibody that was not bound to the protein was washed off. Then, a secondary antibody was diluted at a ratio of 1:1000-1:5000 in a TBST solution containing 5% non-fat skim milk, and the washed filter membrane was incubated in the diluted solution for 2 hours at room temperature. Western blot images were captured using an Amersham Imager 680 (GE Healthcare, Chicago, Illinois, USA).

### Fluorescence Imaging

Samples were placed on clean glass slides, and then fluorescence images were captured using an Amersham Imager 680 (GE Healthcare, Chicago, Illinois, USA).

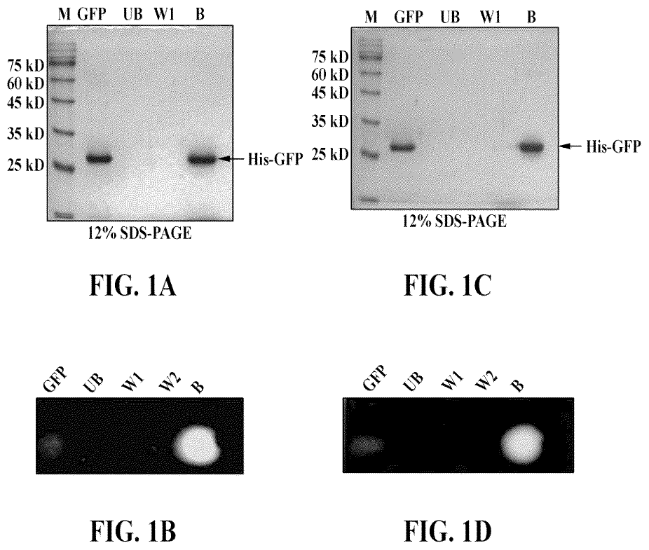

The SDS-PAGE results for Examples 1 and 2 are shown in FIGS. 1A and 1B, and the results of the fluorescence imaging are shown in FIGS. 1C and 1D. In FIGS. 1A to 1D, M represents a molecular weight marker, UB represents the supernatant after centrifugation that is not bound to bentonite, W1 represents the washing solution from the 1^{st} pellet washing, W2 represents the washing solution from the 2^{nd} pellet washing, and B represents an amino acid bound to a bentonite.

Referring to FIGS. 1A and 1B, it can be seen that bentonite and His-GFP bind to form a complex not only at the biological pH of 7.4 but also at a higher pH of 9. In addition, His-GFP was not detected in the supernatant (UB) which contains unbound His-GFP, and was not detected in the washing solution (W1) which is from the 1^{st} pellet washing, indicating that His-GFP was tightly bound to the bentonite.

The results of the fluorescence imaging shown in FIGS. 1C and 1D also show that bentonite and histidine can be stably bound at both pH 7.4 and pH 9.0.

The factors that create the tight bond between bentonite and protein described above, may be predicted. Bentonite is mainly negatively charged, because it acts as a strong cation exchanger matrix, and His-GFP has a net negative charge at pH 7.4, so it is believed that the bond between bentonite and His-GFP is attributed to factors other than the net charge. It is predicted that the binding of His-GFP to bentonite occurs because of the charged surface residues rather than the overall net charge, or because the His-GFP is sandwiched between layered bentonites.

### 3. Experimental Example 2: Lifetime of the Bentonite-Amino Acid Complexes at Different pHs

To determine the lifetime of the bentonite-amino acid complexes at different pHs, the bentonite-amino acid complexes prepared in Examples 1 and 2 were added to Tris-Cl buffer at pH 7.4 and pH 9.0, respectively, and were incubated at 4 °C. The samples were then collected at 24-hour intervals for 72 hours. The collected samples were centrifuged at 5000 G for 5 minutes, and separated into a supernatant(S) and a pellet containing bentonite-amino acid complexes. Then, the pellet was washed once with Tris-Cl buffer. The washed pellet was boiled in SDS sample buffer to separate the amino acid (B) bound to bentonite from the bentonite.

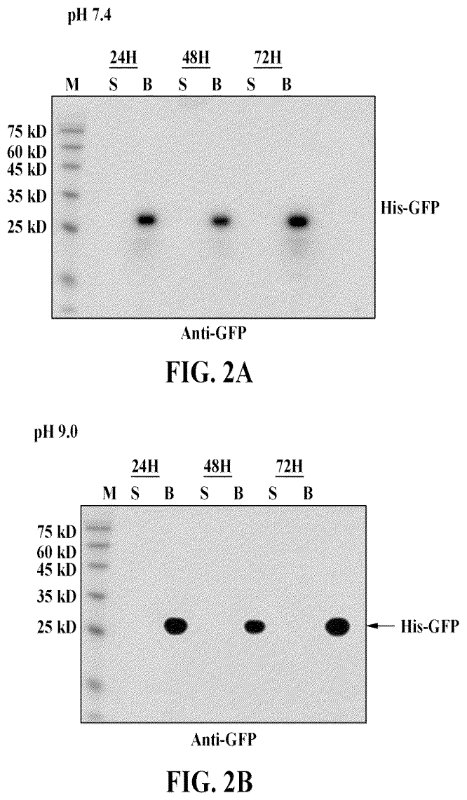

For each sample, the extracted supernatant(S) and the amino acid bound to bentonite (B) were subjected to SDS-PAGE and Western blot analysis using the method that was used in Experimental Example 1 presented above, and the results are shown in FIGS. 2A and 2B. In FIGS. 2A and 2B, M represents a molecular weight marker, S represents a supernatant, and B represents an amino acid bound to bentonite.

Referring to FIGS. 2A and 2B, His-GFP was not detected in the supernatant(S), indicating that the bond between histidine and bentonite was maintained for 72 hours at both pH 7.4 and pH 9.0.

### 4. Experimental Example 3: Lifetime of the Bentonite-Trypsin Complexes Under Gastric Acid

To determine the lifetime of the bentonite-trypsin complex under a gastric acid condition, the bentonite-trypsin complex prepared in Example 3 was added to Tris-Cl buffer at pH 1.2, an adjusted pH that simulates a gastric acid condition, and then samples were collected at 15-minute intervals for 60 minutes at 37 °C. The collected samples were centrifuged at 5000 G for 5 minutes, and separated into a supernatant(S) and a pellet containing bentonite-trypsin complexes. Then, the pellet was washed once with Tris-Cl buffer. The washed pellet was boiled in SDS sample buffer to separate the trypsin (B) bound to bentonite from the bentonite.

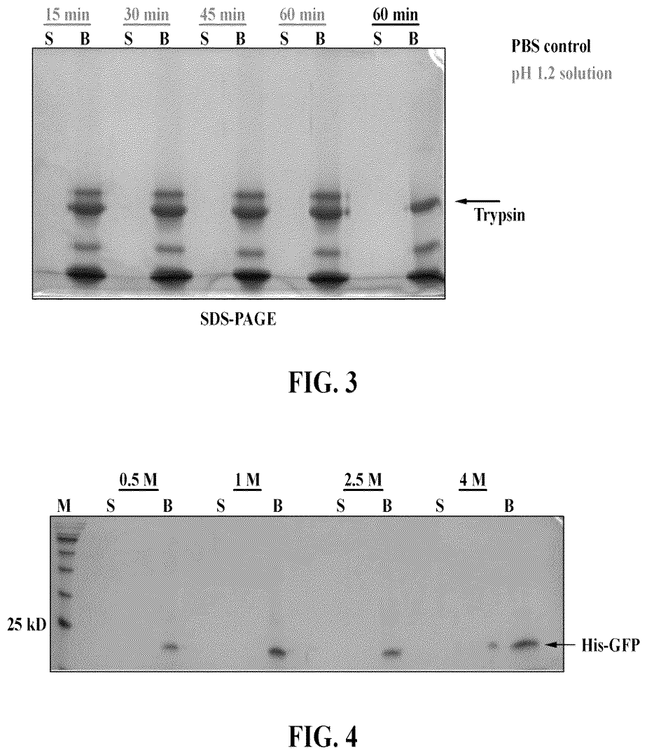

The extracted supernatant(S) and the trypsin bound to bentonite (B) were subjected to SDS-PAGE and Western blot analysis using the method used in Experimental Example 1 presented above, and the results are shown in FIG. 3. In FIG. 3, S represents a supernatant, and B represents a trypsin bound to bentonite.

Referring to FIG. 3, no trypsin was detected in the supernatant(S) for up to 60 minutes, indicating that the bond of the bentonite-trypsin complex was maintained for 60 minutes even under the extreme condition of pH 1.2.

### 5. Experimental Example 4: Effect of Salt Concentration on Bentonite-Amino Acid Complexes

High salt concentrations weaken charge-based interactions between materials. Thus, in order to determine if the bond of the bentonite-amino acid complex was charge-based, we examined whether the bond of the bentonite-amino acid complex was maintained at different salt concentrations.

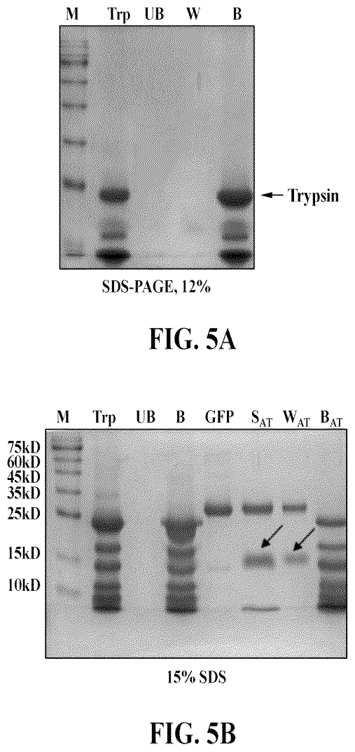

The bentonite-amino acid complex prepared in Example 1 was added to standard PBS buffers (pH 7.4) containing NaCl at concentrations of 0.5 M, 1 M, 2.5 M, and 4 M, respectively, and the mixed solutions were incubated at 4 °C for 8 hours. Then, the supernatants(S) and the pellets were separated by centrifugation at 5000 G for 5 minutes, and the pellets were washed once with PBS buffer. The washed pellets were boiled in SDS sample buffer to separate the amino acid bound to bentonite (B) from the bentonite-amino acid complex. The extracted supernatant(S) and the amino acid bound to bentonite (B) were subjected to SDS-PAGE and Western blot analysis using the method used in Experimental Example 1 presented above, and the results are shown in FIG. 4. In FIG. 4, M represents a molecular weight marker, S represents a supernatant, and B represents an amino acid bound to bentonite.

Referring to FIG. 4, it can be seen that even at a salt concentration of 4 M, no His-GPF was detected in the supernatant, indicating that the bond between bentonite and histidine is well maintained even at high salt concentrations. These results may be interpreted as indicating that the interaction between bentonite and His-GPF is not charge-based. Of note, His-GPF was detected in this experiment at a molecular weight lower than its native molecular weight of 25 kD. It is believed that the high salinity caused an abnormal pattern during gel electrophoresis.

### 6. Experimental Example 5: Maintenance of Enzyme Activity of Trypsin Bound to Bentonite

### (1) Confirming the Binding of Trypsin to Bentonite

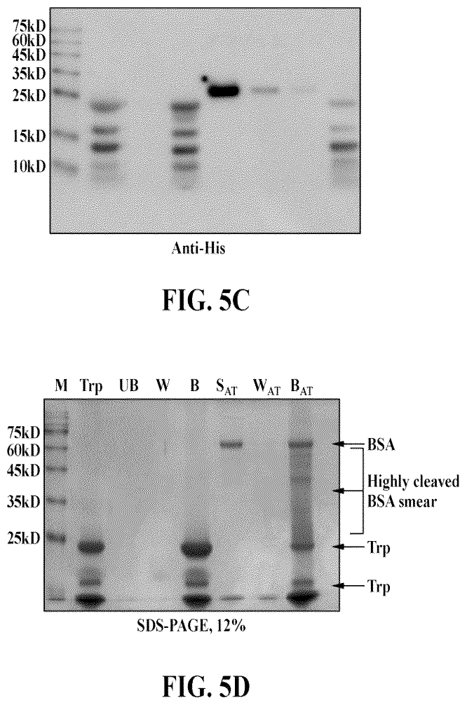

For the bentonite-trypsin complex prepared in Example 3, SDS-PAGE was performed in the way it was performed in Experimental Example 1, and the results are shown in FIG. 5A. In FIGS. 5A to 5D, M represents a molecular weight marker, Trp represents trypsin, UB represents the supernatant containing unbound proteins, W represents the washing solution, and B represents proteins bound to bentonite.

Referring to FIG. 5A, trypsin was detected only in the protein bound to bentonite (B), and was not detected in the washing solution (W) or supernatant (UB), indicating that the trypsin was tightly bound to the bentonite.

### (2) Degradation of His-GFP by Trypsin Bound to Bentonite

The solution containing the bentonite-trypsin complex prepared in Example 3 was mixed with His-GFP and the mixture was incubated at 37 °C for 1 hour. The incubated solution was centrifuged at 5000 G for 5 minutes to separate the solution into a supernatant (S_{AT}) and a pellet. Then, the pellet was washed once with PBS buffer and the washing solution (W_{AT}) was stored for analysis. The washed pellets were boiled in SDS sample buffer to separate the protein bound to bentonite (B_{AT}) from the bentonite-protein complex. For the above extracted sample, 15% SDS-PAGE and Western blot analysis were performed using the method used in Experimental Example 1, and the results are shown in FIGS. 5B and 5C.

Referring to FIGS. 5A to 5D, the SDS-PAGE results (FIG. 5B) show that a unique band of approximately 15 kD was detected in the supernatant (S_{AT}) and the washed solution (W_{AT}) samples. This band represents a fragment of His-GFP cleaved by trypsin, which was not detected in the Western blot analysis results (FIG. 5C) which show that trypsin was non-specifically detected using an anti-His antibody. This result may be interpreted as indicating that the trypsin bound to bentonite retains enzymatic activity.

### (3) Degradation of BSA by Trypsin Bound to Bentonite

The solution containing the bentonite-trypsin complex prepared in Example 3 was mixed with bovine serum albumin (BSA) and then the mixture was incubated at 37 °C for 1 hour. The incubated solution was centrifuged at 5000 G for 5 minutes to separate the solution into a supernatant (S_{AT}) and a pellet. Then, the pellet was washed once with PBS buffer and the washing solution (W_{AT}) was stored for analysis. The washed pellet was boiled in SDS sample buffer to separate the protein bound to bentonite (B_{AT}) from the bentonite-protein complex. For the above extracted sample, 12% SDS-PAGE was performed using the method that was used in Experimental Example 1, and the results are shown in FIG. 5D.

Referring to FIG. 5D, it can be seen that in the bentonite-bound protein (B_{AT}) sample, a blotchy pattern of multiple bands (30-55 kD) was detected, whereas in the supernatant (S_{AT}) and washed solution (W_{AT}) samples this pattern was not detected. This result may be interpreted as meaning that BSA was cleaved by trypsin bound to bentonite, and then the cleaved small fragments were bound to bentonite through interaction with the bentonite.

### (4) X-Ray Diffraction Analysis of Bentonite-Trypsin Complex

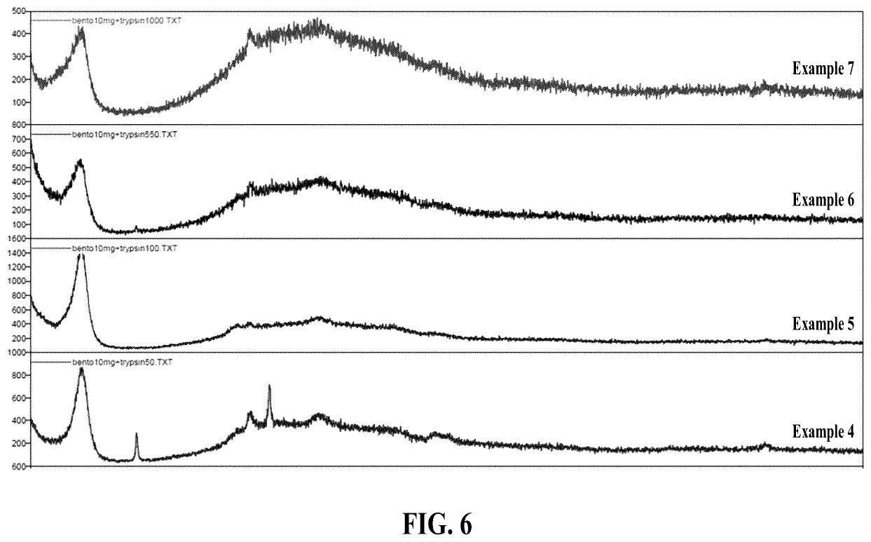

For the bentonite-trypsin complexes prepared in Examples 4 to 7, X-ray diffraction analysis (XRD) was performed, and the results are shown in FIG. 6. Referring to FIG. 6, it can be confirmed that the peak broadens gradually from the peak of Example 4 to the peak of Example 7. That is, it can be seen that trypsin binds to bentonite resulting in an amorphous form, whose peak increases dependent on the amount of the bound trypsin.

### 7. Experimental Example 6: Protein Binding Capacity of Bentonite

### (1) Amount of Trypsin That May Bind to Bentonite

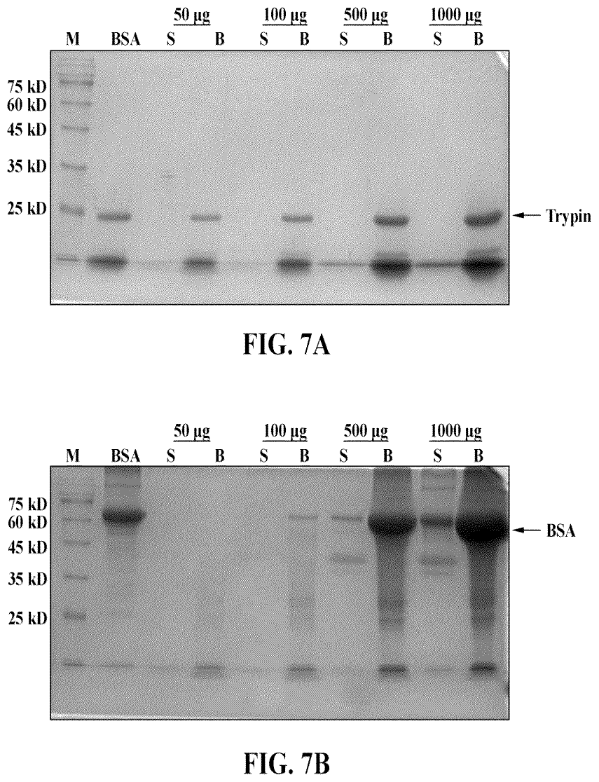

To determine the amount of trypsin that may bind to bentonite, SDS-PAGE was performed on the samples extracted in Examples 4 to 7, and the results are shown in FIG. 7A.

Referring to FIG. 7A, in Example 7 for which 1000 μg of trypsin was added, no trypsin was detected in the supernatant(S), indicating that all of the 1000 μg of trypsin was bound to the bentonite.

### (2) Amount of BSA That May Bind to Bentonite

To determine the amount of BSA that may bind to bentonite, SDS-PAGE was performed on the samples extracted in Examples 8 to 11, and the results are shown in FIG. 7B.

Referring to FIG. 7B, BSA was detected in the supernatants (S) of Example 10 for which 500 μg of BSA was added and Example 11 for which 1000 μg of BSA was added, indicating that all of the 500 to 1000 μg of BSA could not be bound to 10 mg of bentonite.

The above-described result is believed to be due to the fact that trypsin has a protein size of 25 kD, less than half that of BSA (65 kD), and the relatively small molecular size of trypsin allows it to bind to bentonite better than to BSA. Therefore, it may be interpreted that the ability of proteins to bind to bentonite is affected by the size of the protein.

In addition, referring to FIGS. 7A and 7B, it can be seen that the amount of protein (B) released by boiling the bentonite-protein complex is greater for BSA than for trypsin. This difference is presumably due to the fact that trypsin, being a smaller molecule, is more tightly bound between the layers of bentonite compared to BSA, being a larger molecule.

### 8. Experimental Example 7: Confirming the Binding Between Bentonite and Human Serum Albumin

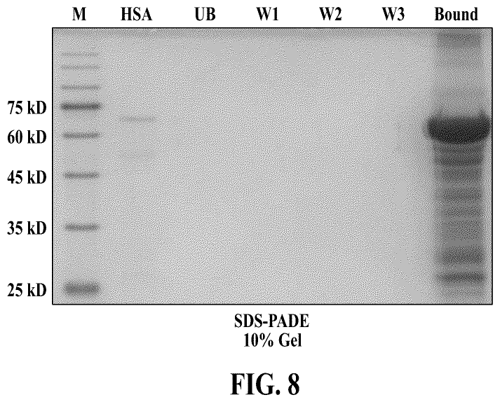

For the bentonite-HSA complex prepared in Example 12, SDS-PAGE was performed in the way it was performed in Example 1, and the results are shown in FIG. 8. In FIG. 8, M represents a molecular weight marker, HSA represents human serum albumin, UB represents the supernatant containing unbound proteins, W1 represents the washing solution from the 1^{st} pellet washing, W2 represents the washing solution from the 2^{nd} pellet washing, W3 represents the washing solution from the 3^{rd} pellet washing, and Bound represents a protein bound to a bentonite.

Referring to FIG. 8, HSA was detected only in the protein bound to bentonite (Bound) region, and was not detected in the washed solution (W) or supernatant (UB) region, indicating that the HSA was tightly bound to the bentonite.

### 9. Experimental Example 8: Confirming That Bentonite Binds to Pepsin

(1) Confirming that bentonite binds to pepsin

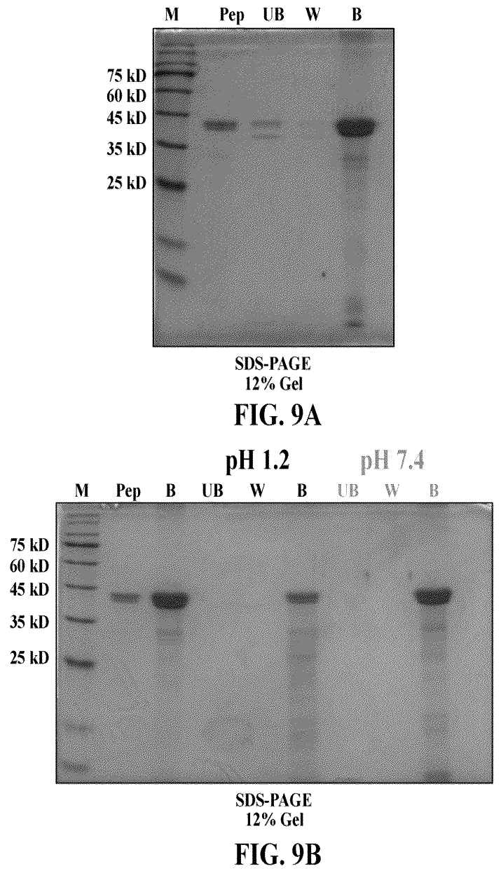

For the bentonite-pepsin complex prepared in Example 13, SDS-PAGE was performed in the way it was performed in Example 1, and the results are shown in FIG. 9A. In FIGS. 9A and 9B, M represents a molecular weight marker, Pep represents pepsin, UB represents the supernatant containing unbound proteins, W represents the pellet washing solution, and B represents proteins bound to bentonite.

Referring to FIG. 9A, pepsin was detected only in the protein bound to bentonite (B) region, and was not detected in the washing solution (W) region, indicating that the pepsin was tightly bound to the bentonite.

### (2) Confirming the Stability of the Bentonite-Pepsin Bond in the Gastrointestinal Environment

For the bentonite-pepsin complex prepared in Example 13, incubation was performed at pH 1.2 for 30 minutes, and then again at pH 7.4 for 30 minutes. For each complex incubated at pH 1.2 and subsequently incubated at pH 7.4, SDS-PAGE was performed in the way it was performed in Example 1, and the results are shown in FIG. 9B.

Referring to FIG. 9B, at both pH 1.2 and pH 7.4, pepsin was detected only in the protein bound to bentonite (B) region, and was not detected in the washing solution (W) region, indicating that the pepsin was tightly bound to the bentonite. This finding may be interpreted as meaning that the bentonite-pepsin complex may remain stably bound in the gastrointestinal environment.

While the embodiments are described with reference to drawings, it will be apparent to one of ordinary skill in the art that various alterations and modifications in form and details may be made in these embodiments without departing from the spirit and scope of the claims and their equivalents. For example, suitable results may be achieved if the described techniques are performed in a different order and/or if components in a described system, architecture, device, or circuit are combined in a different manner and/or replaced or supplemented by other components or their equivalents.

Therefore, other implementations, other embodiments, and equivalents to the claims are also within the scope of the following claims.

What is claimed is:

1. A bentonite-amino acid complex comprising:
a bentonite; and
an amino acid,
wherein the complex is formed by immobilizing the amino acid on the bentonite, and the amino acid is a hydrophilic amino acid.

2. The bentonite-amino acid complex of claim 1, wherein the amino acid comprises any one or more amino acids selected from the group consisting of arginine, histidine, lysine, aspartic acid, glutamic acid, serine, threonine, asparagine, glutamine, cysteine, glycine, and proline.

3. The bentonite-amino acid complex of claim 1, wherein the bentonite-amino acid complex maintains a bond between the bentonite and the amino acid at pH 1 to pH 10 for more than 72 hours.

4. The bentonite-amino acid complex of claim 1, wherein the bentonite-amino acid complex maintains a bond between the bentonite and the amino acid for 8 hours at a salt concentration of 4.0 M or less.

5. A bentonite-protein complex comprising:
a bentonite; and
a protein,
wherein the complex is formed by immobilizing the protein on the bentonite, and the protein is a protein comprising histidine.

6. The bentonite-protein complex of claim 5, comprising no more than 2000 μg of the protein per 10 mg of the bentonite.

7. The bentonite-protein complex of claim 5, wherein the protein comprises any one or more proteins selected from the group consisting of trypsin, pepsin, human serum albumin and bovine serum albumin (BSA).

8. The bentonite-protein complex of claim 7, wherein the protein is trypsin, and the trypsin is comprised in an amount of no more than 2000 μg per 10 mg of the bentonite.

9. The bentonite-protein complex of claim 5, wherein the bentonite-protein complex maintains a bond between the bentonite and the protein at pH 1 to pH 10 for more than 72 hours.

10. The bentonite-protein complex of claim 5, wherein the bentonite-protein complex maintains a bond between the bentonite and the protein for 8 hours at a salt concentration of 4.0 M or less.

11. The bentonite-protein complex of claim 7, wherein the protein is trypsin or pepsin, and the bentonite-protein complex is available as a digestive enzyme.

12. A method of preparing a bentonite-protein complex, the method comprising:
washing a bentonite;
preparing a buffer containing a protein;
mixing the bentonite with the buffer to bind the bentonite and the protein; and
centrifuging the mixed solution to separate unbound proteins.

13. The method of claim 12, wherein the protein is a protein comprising histidine.

14. The method of claim 12, wherein the protein comprises any one or more proteins selected from the group consisting of trypsin, pepsin, human serum albumin and bovine serum albumin (BSA).
